(19) United States
(12) Patent Application Publication
(10) Pub. No.: US 2009/0105642 A1
(43) Pub. Date: Apr. 23, 2009

(54) LOW PROFILE AGENT DELIVERY PERFUSION CATHETER HAVING REVERSIBLY EXPANDING FRAMES

(75) Inventors: Michael J. Leonard, Olympia, CA (US); William E. Webler, Escondido, CA (US)

Correspondence Address: FULWIDER PATTON, LLP (ABBOTT), 6060 CENTER DRIVE, 10TH FLOOR, LOS ANGELES, CA, 90045, US

(73) Assignee: ABBOTT CARDIOVASCULAR SYSTEMS INC., Santa Clara, CA (US)

(21) Appl. No.: 11/876,481

(22) Filed: Oct. 22, 2007

(51) Int. Cl. A61M 25/10

(52) U.S. Cl. 604/103.09

(57) ABSTRACT

An agent delivery catheter and method, configured to deliver an agent to an inner surface of a patient's body lumen by forming an agent containment pocket along the inner surface of the patient's body lumen wall between a proximal expandable frame and a distal expandable frame, while minimizing ischemic conditions during the procedure.

### CROSS-REFERENCES TO RELATED APPLICATIONS

None

### BACKGROUND OF THE INVENTION

The present invention relates generally to medical devices, and more particularly to a catheter for delivery of an agent to the coronary or peripheral vasculature.

In the treatment of diseased vasculature, therapeutic agents have commonly been administered, typically as part of other interventional therapies such as angioplasty or stent delivery. Local, as opposed to systemic delivery is a preferred method of treatment in that smaller total levels of medication are administered in comparison to systemic dosages yet are concentrated at a specific site. As a result, local delivery produces fewer side effects and achieves more effective results.

A variety of methods and devices have been proposed for percutaneous drug delivery to a diseased region of the vasculature. For example, catheters having porous balloons can be used to deliver a therapeutic agent infused into the inflatable interior of the porous balloon and through the porous wall of the balloon. Alternatively, prostheses such as stents or other implantable devices provide for local drug delivery when coated or otherwise made to include a therapeutic agent which elutes from the implanted prosthesis. Another suggested method involves the use of one or more catheters having multiple balloons. The diseased region is isolated by inflating the balloons on either side of the diseased region, and the therapeutic agent is infused through a lumen of the catheter shaft and into the isolated diseased region from a delivery port on the catheter shaft located between the balloons. However, the balloons inflated against the vessel wall occlude the vessel, and thus create ischemic conditions there along and distal thereto.

In order to properly position the distal end of a drug delivery catheter in a patient's tortuous distal vasculature, the catheter should preferably have a low-profile, flexible distal section despite also having the necessary structural components required for the drug delivery at the operative distal end of the catheter. One difficulty has been providing for a large amount of drug taken-up and retained at the diseased site, while minimizing the wash out of significant amounts of drug downstream of the treatment site. Drug wash out reduces the efficiency of local intravascular drug delivery, in addition to causing potentially harmful systemic exposure to the drug. Therefore, it would be a significant advance to provide an improved device and method for providing therapy to a desired location within a patient's body lumen.

### SUMMARY OF THE INVENTION

The invention is directed to an agent delivery catheter and method configured to deliver an agent to an inner surface of a patient's body lumen by forming an agent containment pocket along the inner surface of the body lumen wall, while minimizing ischemic conditions during the procedure.

A catheter of the invention generally includes an elongated shaft having an agent delivery lumen which is in fluid communication with an agent delivery distal port, and a distal shaft section which has a proximal expandable frame and a distal expandable frame, and a tubular membrane extending from the proximal to the distal frame. The tubular membrane has a proximal end section secured to the proximal frame, a distal end section secured to the distal frame, a central section therebetween, an outer surface, and an inner surface defining a perfusion channel extending through the membrane. The shaft extends in the perfusion channel, and the agent delivery lumen extends across the membrane (e.g., to a port in the sidewall of the membrane) from the inner towards the outer surface thereof, so that the catheter is configured to deliver an agent to an agent containment pocket which extends along the inner surface of the patient's body lumen wall in the space between the inner surface of the patient's body lumen wall and an outer surface of the central section of the membrane with the frames in a radially expanded configuration.

Each frame preferably has a plurality of struts in a network configured to radially self-expand when an radially restraining force is removed from the frame. A maximum diameter section of each frame is secured to the membrane, such that the expanded frames seal the membrane against the vessel wall, to thereby contain the agent infused from the agent lumen into the agent containment pocket extending along the central section of the catheter membrane. Although the agent containment pocket thus isolates the region of the patient's vessel from blood flow, the perfusion channel allows for blood to continue flowing in the patient's body lumen through the frames.

The perfusion channel is configured to have a relatively large inner diameter. Specifically, the relatively thin wall thickness of the membrane and the struts of the frame provide the perfusion channel therein with a large inner diameter. Moreover, the shaft extending in the perfusion lumen does not have to define more than one fluid delivery lumen, namely the agent delivery lumen, such that the shaft provides both a large agent delivery lumen and a low profile. Consequently, with the frames in the radially expanded configuration, blood flow in the patient's body lumen is free to perfuse through the center of the sealing frames with little or no constriction. Thus, the catheter minimizes ischemic conditions caused by its use in delivering agent to the vessel wall, and allows for extended treatment durations of over an hour.

The frames repeatedly radially expand to a set expanded configuration into contact with the patient's blood vessel wall without a risk of over-expanding or otherwise producing a potentially variable expanded configuration unlike a balloon. The frames thus provide a durable support structure for the membrane that provides for ease of deployment of the catheter in the patient's body lumen and avoids damaging the wall of the patient's blood vessel. In contrast, isolating a treatment region in a patient's blood vessel using a catheter having two longitudinally spaced apart balloons can be problematic because the balloons have to be inflated. Such balloons can potentially over expand and damage the blood vessel, and may axially elongate during inflation so that the size of the containment region between the two balloons is not as readily controlled. Moreover, inflated occlusion balloons occlude the body lumen and thus provide no perfusion, or relatively little perfusion if a perfusion lumen is added to the catheter shaft.

Additionally, the catheter is preferably configured such that the resulting agent containment pocket extends fully around the inner circumference of the body lumen. Thus, with the frames expanded against the vessel wall, the catheter allows for delivering agent to the entire circumference and length of the vessel wall extending between the deployed sealing members. As a result, the catheter maximizes the amount of the vessel wall directly exposed to the agent, and, moreover, thus minimizes the duration required for the procedure (while nonetheless allowing for the procedure to be done over longer periods of time due to the non-occlusive nature of the catheter of the invention).

A method of the invention generally involves introducing within a patient's body lumen a catheter comprising an elongated shaft having an agent delivery lumen, a proximal expandable frame and a distal expandable frame on a distal shaft section, and a tubular membrane which has a proximal section secured to the proximal frame, a distal section secured to the distal frame, a central section therebetween, an outer surface, and an inner surface defining a perfusion channel therethrough, the perfusion channel having the shaft extending therethrough and the agent delivery lumen extending across the membrane from the inner towards the outer surface thereof, typically to a single agent delivery port in a sidewall of the membrane, such that agent can be delivered along an outer surface of the membrane. The method includes radially expanding the frames against an inner surface of the body lumen wall, and delivering an agent to an agent containment pocket which extends along the inner surface of the patient's body lumen wall along an outer surface of the central section of the membrane. In one embodiment, one of the proximal or distal frames has a lower radially expansive force than the other frame to thereby release agent above a seal-breaking pressure, such that the method includes preventing or reducing over pressurization of the patient's body lumen by releasing agent from the agent containment pocket.

A variety of suitable agents, including diagnostic and therapeutic agents, can be delivered to the agent containment pocket using the catheter and method of the invention. The agent is typically a therapeutic agent for restenosis, although the agent can be delivered for a variety of treatment procedures, including treatment of a diseased (occluded) blood vessel by delivery of the agent directly into the diseased blood vessel, or treatment of the myocardium of the heart by delivery of an agent into one of the (healthy) coronary arteries. In a presently preferred embodiment, the agent is an anti-inflammatory agent including steroids, or is an agent that induces cholesterol efflux from arterial wall plaque including ApoA1 mimetic peptides, PPARα agonists.

A catheter of the invention allows for improved delivery of an agent to a patient's vessel wall by allowing for agent delivery to take place over a desired period of time while allowing for blood to perfuse through a large perfusion channel and thereby prevent or minimize disadvantageous, damaging ischemia in the vessel wall. Despite having a large perfusion channel and agent delivery lumen, the catheter has a highly flexible distal section with a low-profile collapsed configuration, which facilitates advancement and retraction in the patient's body lumen. Moreover, the catheter preferably maximizes agent up-take in the vessel wall, while nonetheless minimizing wash out of the agent in the blood vessel. These and other advantages of the invention will become more apparent from the following detailed description of the invention and accompanying exemplary drawings.

### BRIEF DESCRIPTION OF THE DRAWINGS

FIG. 1 is an elevational, partially in section, view of an agent delivery perfusion catheter embodying features of the invention, having proximal and distal frames in a collapsed configuration within a patient's body lumen.

FIG. 2 illustrates a perspective view of the catheter of FIG. 1 with the frames in an expanded configuration.

FIG. 3 is an elevational view, partially in section, of the catheter of FIG. 1 with the frames in an expanded configuration in a patient's body lumen during delivery of an agent.

FIGS. 4-7 are transverse cross sections of FIG. 3, taken along lines 4-4, 5-5, 6-6, and 7-7, respectively.

FIG. 8 illustrates an alternative embodiment, in which the frames share a common central skirt slidably mounted on the catheter shaft.

FIG. 9 illustrates an alternative embodiment, in which each frame has a free distal end.

FIG. 10 is a transverse cross section of FIG. 9, taken along line 10-10.

### DETAILED DESCRIPTION OF THE PREFERRED EMBODIMENTS

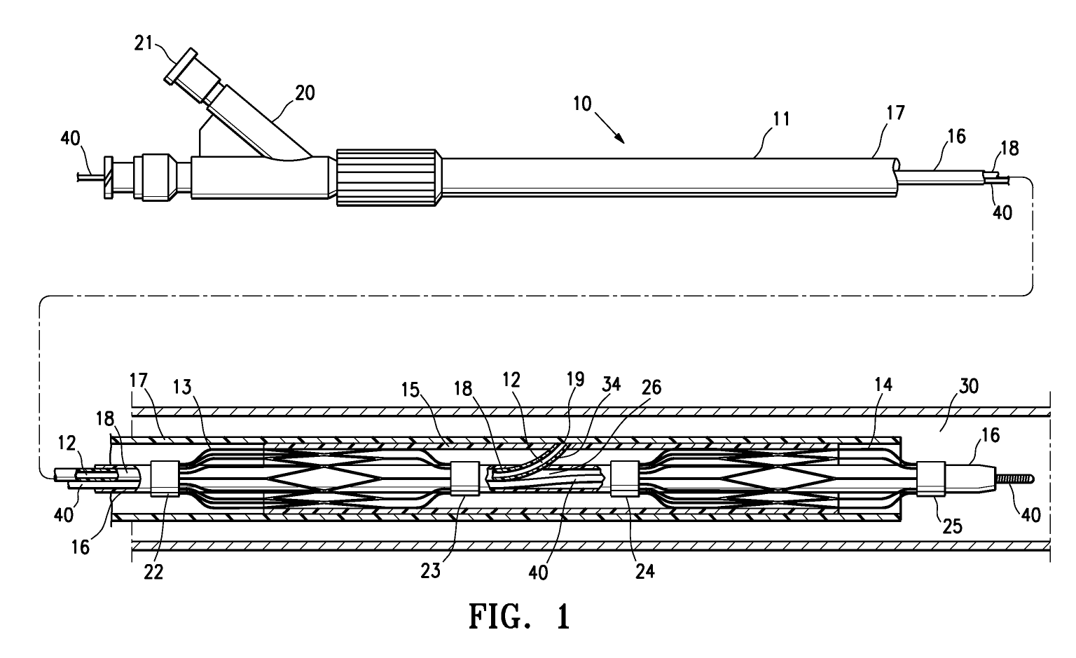

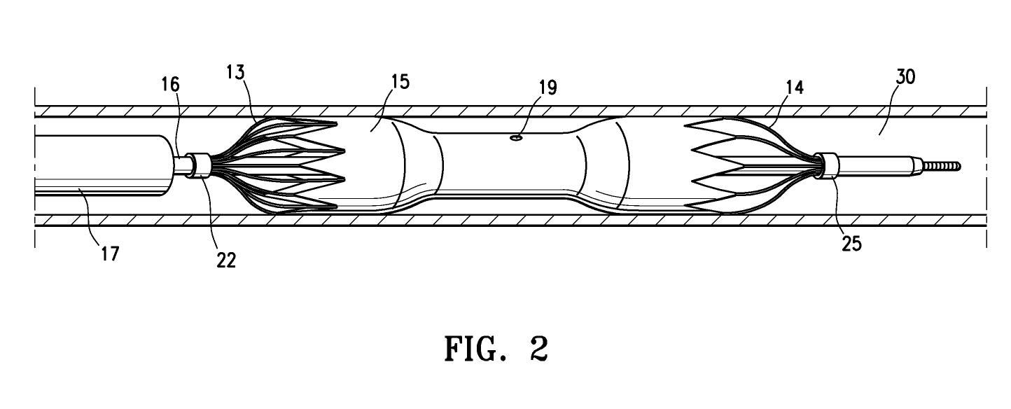

FIG. 1 illustrates an elevational, partially in section, view of an agent delivery perfusion catheter 10 embodying features of the invention, generally comprising an elongated shaft 11 having an agent delivery (infusion) lumen 12 in fluid communication with an agent delivery distal port 19 at the distal end of the agent delivery lumen 12, a proximal frame 13, a distal frame 14, and a tubular membrane 15 extending from the proximal frame 13 to the distal frame 14. The tubular membrane 15 has a proximal section secured to the proximal frame and a distal section secured to the distal frame, and a central section therebetween. The frames reversibly expand from a low profile configuration to a radially expanded configuration. FIG. 1 illustrates the frame 13, 14 in a low profile collapsed configuration, and FIG. 2 illustrates a perspective view of the catheter of FIG. 1 with the frames 13, 14 in the radially expanded configuration. Although the collapsed frames 13, 14 and membrane 15 are illustrated in FIG. 1 radially spaced from the underlying section of the shaft 11 for ease of illustration, it should be understood that the frames 13, 14 and membrane 15 typically collapse down onto the shaft in the collapsed configuration.

In the illustrated embodiment the shaft 11 comprises an inner tubular member 16, and an outer sheath member 17 slidably disposed on the inner tubular member. The frames 13, 14 are fixedly secured to the inner tubular member 16, and are configured to radially self-expand to an expanded configuration by release of a radially restraining force, which in the illustrated embodiment is provided by the shaft outer member 17. Thus, the frames 13, 14 are biased to automatically radially expand to the expanded configuration by slidably displacing the inner tubular member 16 and the outer sheath member 17 relative to one another, such that the frames 13, 14 deploy upon becoming distally spaced from the distal end of the outer sheath member 17. The frames are typically deployed to the expanded configuration by proximally retracting the outer sheath member 17 while holding the inner member 16 stationary to maintain the position of the frames within the body lumen 30. Although less preferred, due in part to the potential for damage to the vessel wall, the inner member 16 can alternatively or additionally be advanced distally during deployment of the frames 13, 14. In one embodiment, the outer sheath member 17 is configured to also recover the expanded frames by advancing distally over the expanded frames, to thereby re-collapse the frames in the lumen of the outer sheath member 17 for repositioning or removal of the device from the patient's body lumen. In an alternative embodiment, the outer sheath member 17 is configured to be peeled or otherwise removed from the inner tubular member 16 during deployment of the frames 13, 14, and a separate recovery catheter (not shown) is slidably advanced over the shaft 11 to collapse the frames for recovery. For example, a removable outer sheath member 17 typically has a weakened wall portion extending along the length thereof, so that as the outer sheath member 17 is proximally retracted it is caused to peel off the inner tubular member 16 at the proximal end of the catheter 10.

In the illustrated embodiment, the inner member 16 is configured to be slidably advanceable over a guidewire 40 for positioning the catheter 10 in the patient's body lumen 30, such that the inner tubular member 16 has a guidewire lumen 26 separate from the agent delivery lumen 12. The inner tubular member 16 typically tapers to a smaller diameter distal to the distal frame 14, providing a low profile distal end. In the illustrated embodiment, the agent delivery lumen 12 is defined by a tube 18 within the inner tubular member 16. However, a variety of suitable shaft designs can be used as are commonly known, including dual lumen extrusions. In one alternative embodiment, the shaft is provided with the general support and pushability required by a fixed core wire with a floppy distal tip, fixed to the shaft inner member 16 from the proximal to the distal end of the shaft inner member 16, to thereby facilitate advancing the catheter 10 in the patient's tortuous vasculature. In one embodiment, the distal frame 14, located distal to the agent delivery lumen 12, is mounted on a core wire and not on a lumen-defining portion of the shaft 11, further reducing the profile of the shaft with a corresponding increase in the area of the perfusion channel 31. However, a variety of suitable shaft configurations can alternatively be used which generally provide an agent delivery lumen and an advanceable shaft for supporting the frames 13, 14.

A proximal adapter 20 on the proximal end of the catheter 10 provides access to the guidewire lumen 26, and has a port 21 which is in fluid communication with the agent delivery lumen 12 and which is configured for connecting to a fluid agent source (not shown). The adapter can be configured to facilitate displacing the outer member 17 relative to the inner member 16 to deploy the frames 13, 14 (primarily in embodiments in which the outer sheath member 17 is not designed to be removed from the inner member 16), similar to conventional adapters or handles on self-expanding embolic protection filters and stent delivery systems.

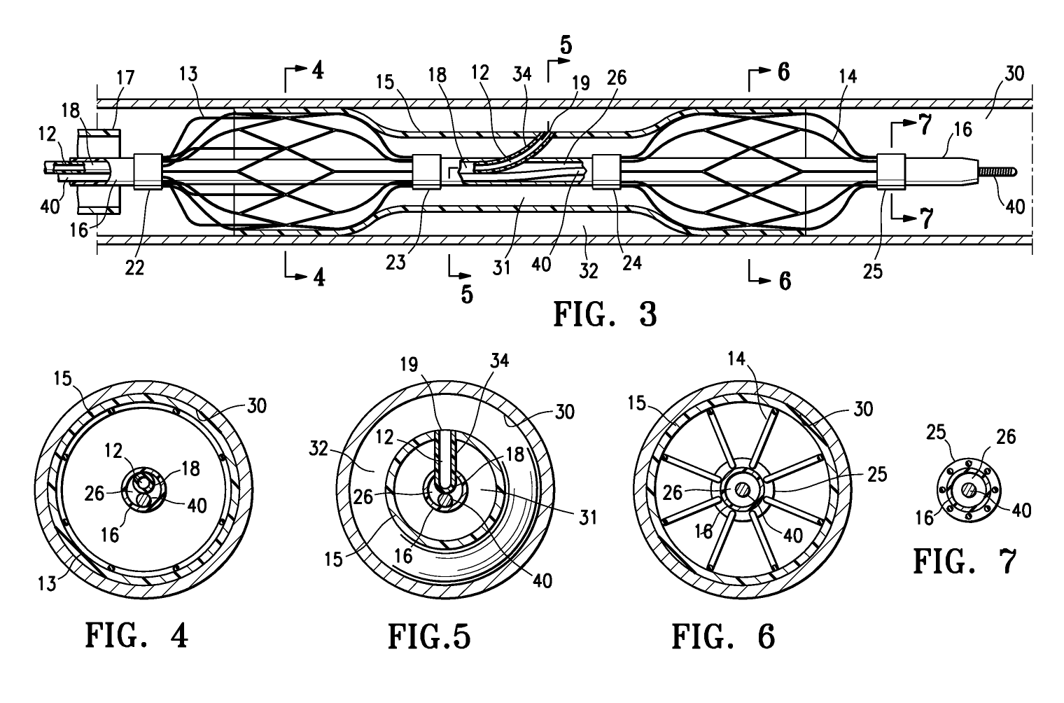

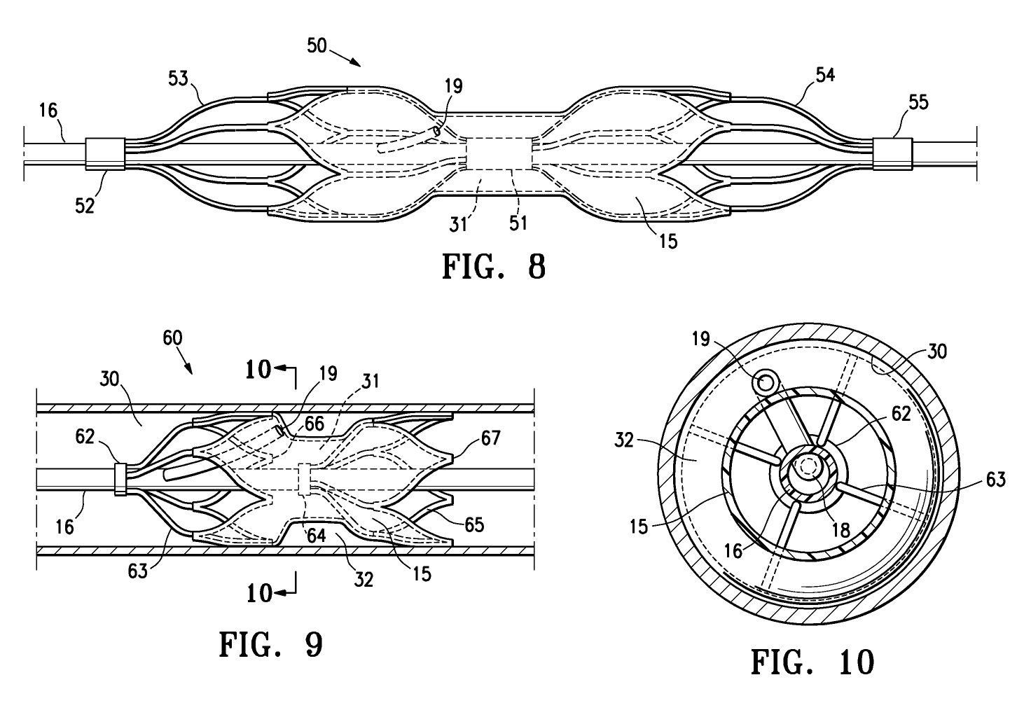

Each frame 13, 14 has proximal end, a distal end, a plurality of struts forming a sealing body with a conical or tapering end section, and at least one skirt section mounting the frame on the distal shaft section. In the embodiment of FIG. 1, each frame 13, 14 has a proximal and a distal skirt section mounting the frame on the inner tubular member 16. Specifically, the proximal frame 13 has an annular proximal skirt section 22 and an annular distal skirt section 23, and the distal frame 14 has an annular proximal skirt section 24 and an annular distal skirt section 25, and the struts forming the expandable portion of the each frame extend from the proximal to the distal skirt section of the frame. Thus, in the expanded configuration the sealing body of the frame is between conical proximal and distal ends extending down to the skirt sections of the frame (see FIGS. 2 and 3). In the embodiment of FIG. 1, the generally tubular sealing body of each frame is formed by multiple undulating rings, with longitudinal mounting struts extending proximally and distally therefrom forming the conical ends of the frame. However, the body and/or ends of the frames can have various configurations (see e.g., the embodiments of FIGS. 8 and 9). The conical end section of each frame typically has at least four mounting struts, although fewer (e.g., at least two) or more mounting struts can alternatively be used. Thus, a variety of suitable frame configurations can be used in a catheter of the invention, although a preferred frame has a self-expansive force sufficient to fully open the frame to its maximum radially expanded outer diameter without requiring further radially expansive force to be applied to the frame.

The membrane 15 is bonded to the body of the frame typically by heat bonding although an adhesive could additionally or alternatively be used. In the illustrated embodiment, the membrane 15 is bonded to the struts forming the undulating rings of the frame and is preferably not bonded to the distal mounting struts of the proximal frame 13 or the proximal mounting struts of the distal frame 14 as discussed in more detail below. The ends of the membrane have a zigzag shape corresponding to the undulating ring of the frame bonded thereto in the illustrated embodiment (see FIG. 2), although one or both ends of the membrane can alternatively have a substantially circular shape (e.g., extending continuously around the frame circumference in a plane perpendicular to the longitudinal axis of the frame). Although not illustrated in FIGS. 4 and 6, in one embodiment, the heat bonding melts the membrane, causing it to flow around the struts of the frame and bond to itself, thus encapsulating the struts.

From the collapsed configuration, the network of struts articulate to expand the tubular body of the frame radially in all directions (i.e., around the entire circumference of the frame) to the expanded diameter. To allow the frame to expand and collapse, one of the annular skirt sections, typically the distal skirt section 23, 25, is slidably mounted on the inner member 16, and the other skirt section (e.g., the proximal skirt section 22, 24) is fixedly mounted to the inner member 16. Thus, the distal skirt sections 23 and 25, typically comprising a polymeric or metal ring, will slide distally on the inner member 16 as the frames 13, 14 radially collapse from the expanded configuration. Fixedly (i.e., non-moveably bonding) mounting one of the skirt sections of the frame to the shaft can be achieved using a variety of suitable configurations and methods including adhesively bonding the mating surfaces. Although illustrated as a ring member, the skirt section should be understood to refer to a variety of suitable structural configurations which mount the frame struts on the shaft, including directly bonding the struts thereto.

FIG. 3 illustrates the catheter of FIG. 1 deployed in a patient's body lumen 30 during delivery of an agent to the wall of the body lumen 30, and FIGS. 4-7 illustrate transverse cross-sectional views of the catheter of FIG. 3, taken along lines 4-4, 5-5, 6-6, and 7-7, respectively. The frames 13, 14 expand against the patient's body lumen wall, and an inner surface of the tubular membrane 15 defines a perfusion channel 31. Specifically, with the outer member 17 retracted such that the frames 13, 14 are fully radially expanded in a patient's blood vessel, blood flow in the vessel is able to flow past the deployed frames 13, 14 through the perfusion channel 31 within the membrane 15. The membrane collapses with the frames 13, 14 to a collapsed configuration so that the membrane is positioned within the outer member 17 and the perfusion channel 31 is closed when the outer member 17 is in the advanced configuration.

The membrane 15 central section is radially spaced from the underlying conical end sections of the frames 13, 14 of the embodiment of FIG. 1, such that the perfusion channel 31 has an inner diameter along the central section of the membrane that is larger than an outer diameter of the frame conical end sections extending therealong. In FIG. 3, the membrane 15 is illustrated in longitudinal cross section, showing the inner member 16 of the shaft 11 extending within the perfusion channel 31. The shaft inner member 16 is configured to have a low profile, thus having minimal impact on the useful area of the perfusion channel 31. Preferably, the shaft inner member 16 is coaxially centered within the expanded membrane 15. Although less preferred due to the potential to compromise the sealing profile, the shaft inner member 16 can alternatively be positioned eccentrically within the lumen of the tubular membrane 15, as for example in an embodiment (not shown) in which the shaft extends along and in contact with (e.g., secured to) the inner surface of the tubular membrane 15.

With the frames 13, 14 in the deployed configuration in the patient's body lumen 30, an agent containment pocket 32 is formed along the inner surface of the wall of the body lumen 30 between the deployed frames 13, 14. The agent containment pocket 32 is formed by the outer surface of the membrane 15. Specifically, the membrane 15 is connected to the frames such that radially self-expanding the frames is configured to open the perfusion channel 31 through the membrane, position the maximum diameter tubular body of the frames against the inner surface of the body lumen wall to prevent or inhibit blood flow along the outer surface of the membrane, and to position the outer surface of the central section of the membrane radially spaced from the inner surface of the patient's body lumen wall to form the agent containment pocket 32. The membrane is typically contoured to have a smaller outer diameter along the central section of the membrane than along the proximal and/or distal sections of the membrane in the expanded configuration.

The agent delivery distal port 19 allows for flowing an agent from the lumen 12 into the agent containment pocket 32. Specifically, the agent delivery lumen 12 extends across the membrane 15 from the inner surface toward the outer surface of the central section of the membrane 15 (e.g., to agent delivery port 19 in a sidewall of the membrane 15). In the illustrated embodiment, a short conduit 34 defining a distal end section of the agent delivery lumen 12 extends at an angle radially away from the shaft 11, which may be secured to the distal end of tube 18 or an integral distally extending end portion of tube 18. However, a variety of suitable configurations can be used, including one or more agent delivery ports on an outer surface of the shaft 11 in the embodiment in which the shaft inner member 16 is eccentrically positioned within the membrane 15 and secured to an inner surface thereof. Although the illustrated embodiment has a single agent delivery port 19, the shaft 11 can be provided with multiple agent delivery ports and/or agent delivery lumens in alternative embodiments (not shown). The catheter 10 can thus be configured for delivery of a single agent, or for sequential or simultaneous delivery of multiple agents.

In a method of delivering an agent to the patient's body lumen 30, the catheter 10 is introduced within the patient's body lumen 30. Once the catheter distal section is at the desired location in the body lumen, each frame 13, 14 is radially self-expanded. For example, outer member 17 is preferably proximally retracted while the inner member 16 is held stationary, although the inner member 16 can alternatively be distally advanced out the distal end of the outer member 17, to remove the radially restraining force of the outer member 17 from the frames 13, 14. The distal frame 14 will deploy first as the outer member is proximally retracted therefrom, and then the proximal frame 13 will deploy as the outer member is further proximally retracted. The deployed frames extend fully around the inner circumference of the body lumen 30, as best illustrated in FIGS. 4 and 6, to exert a sealing force against the body lumen wall, and may dilate/expand the body lumen wall, although the frames are preferably configured to exert a radial force that does not cause harm or otherwise promote restenosis at the treatment location. Irrespective of the shape and contour of the inner surface of the patient's body vessel, the frames seal against the inner surface, including curved or non-circular sections of the vessel. The catheter 10 can be provided in a range of sizes depending on the patient's body vessel size and intended use of the catheter 10, which would vary the length of the agent containment pocket 32 and/or the expanded outer diameter of the frames 13, 14. Because the frames radially expand to a set, known diameter without the potential of over or under expanding, the deployment procedure is facilitated, to provide affective sealing for the agent containment pocket and a large perfusion channel.

With the frames 13, 14 thus deployed in the body lumen 30, an agent is delivered to the agent containment pocket 32. The agent flows from the agent delivery lumen 12, and out the port 19, and is held by the membrane 15 in the agent containment pocket 32 so that the agent will absorb into or otherwise treat or act upon the wall of the body lumen 30. In one embodiment, the agent flow is started before outer member 17 is retracted from the proximal frame 14 or from the distal frame 13 in order to flush the agent containment pocket 32 with agent (e.g., displace blood/body fluid with agent). In an alternative embodiment, the agent flow is started after both frames are expanded, to prevent or minimize the systemic release of the agent. In a retrograde insertion in which the blood is flowing from the distal towards the proximal frame in the blood vessel, the agent flow through the agent delivery lumen 12 is typically started before the proximal frame 13 is fully expanded against the body lumen wall in order to flush the agent containment pocket 32 with agent. In one embodiment, a second membrane (not shown) having a porous wall is provided on top of the agent containment pocket membrane 15 to limit the loss of agent to branching vessels (not shown) in the isolated region of the patient's vessel wall. Such an outer porous membrane is configured to slow the flow of agent out of the agent containment pocket 32 into side branch vessels, to increase the amount of agent that penetrates the adjacent sections of the wall of the patient's body lumen 30.

After a procedure in the patient's body lumen 30, the frames 13, 14 are collapsed in a recovery catheter which is either a separate recovery catheter (not shown) or the outer sheath member 17. The recovery catheter has a distal recovery section configured to be slid over the expanded frames in the body lumen to thereby collapse the frames for repositioning or removal of the catheter 10 from the body lumen. Thus, the catheter 10 is fully retrievable and allows for extended but temporary drug delivery to an isolated region of the patient's body lumen.

In the embodiment of FIG. 1, the distal skirt 23 of the proximal frame 13 is proximally spaced from the proximal skirt 24 of the distal frame 14. FIG. 8 illustrates an alternative embodiment in which the proximal and distal frames of device 50 share a common intermediate skirt 51. Specifically, the device 50 has a proximal frame 53 and a distal frame 54, and the intermediate skirt 51 therebetween is slidably mounted on the shaft inner tubular member 16. The proximal skirt 52 of the proximal frame 53 is fixedly mounted to the shaft inner tubular member 16 similar to skirt 22 of frame 13 of the embodiment of FIG. 1. The distal frame 54 has a slidably mounted annular distal skirt 55 similar to annular distal skirt 25 of distal frame 14 of the embodiment of FIG. 1.

It should be understood that the proximal and distal frames 53, 54 can be formed of a continuous member, such that the mounting struts extending from either end of the intermediate skirt 51 are integral and continuous through the intermediate skirt 51. Alternatively, the mounting struts extending from either end of the intermediate member are separate struts which are joined together by the common intermediate skirt 51.

In the embodiment of FIG. 8, the frames 53, 54 each have a single undulating ring structure which radially expands against the inner surface of the wall of the patient's body lumen 30, with four longitudinally extending mounting struts forming the conical end sections of the frames. However, a variety of suitable frame configurations can be used including multiple undulating ring structures such as in the embodiment of FIG. 1. Similar to the embodiment of FIG. 1, the membrane 15 preferably bonded, e.g., heat bonded, to the undulating ring of the frame without bonding to the mounting struts which extend longitudinally to the intermediate skirt 51, such that the perfusion channel 31 has an inner diameter along the agent containment pocket that is larger than an outer diameter of the frame 53, 54 conical end sections extending therealong.

Although the frames of devices 10 and 50 each have conical proximal and distal sections formed by mounting struts extending to skirt sections of the frames, in alternative embodiments, one or both of the proximal and distal frames has a free distal end, similar to a self-expanding stent, such that the distal end of the frame is not attached to the inner tubular member 16. FIG. 9 illustrates an embodiment in which device 60 has a proximal frame 63 with a free distal end 66 and a proximal skirt 62 fixedly mounted to the shaft inner tubular member 16, and a distal frame 64 with a free distal end 67 and a proximal skirt 64 fixedly mounted to the shaft inner tubular member 16. FIG. 10 is a transverse cross sectional view of the catheter 60 of FIG. 9, taken along lines 10-10. The free ends of each frame 63, 64 allow the frame to essentially elongate during radial collapse of the frames 63, 64 into the recovery catheter. As a result, when the proximal frame is collapsing, it naturally promotes the capturing and folding of the membrane inwardly, which facilitates advancing the recovery catheter to capture the device 60. Additionally, the embodiment of FIG. 9 facilitates providing a very low profile device. Although guidewire 40 is not illustrated within shaft inner member 16 adjacent to agent delivery lumen tube 18 in FIG. 10, it should be understood that the shaft inner member 16 can be configured to slidably advance over guidewire 40 similar to the embodiment of FIG. 1. Alternatively, the proximal skirts of each frame 63, 64 can be mounted onto a shaft that is formed by a core wire, with an agent delivery lumen tube similar to tube 18 extending along the core wire.

Each frame of devices 10, 50 and 60 is configured to radially expand against the vessel wall, although the expansion characteristics of the frames can be varied, for example to expand to different diameters or with different amounts of radially expansive force. In general the radial outward force of each frame (proximal and distal) can be tuned by adjusting the wall thickness of the struts. These frame radial outward force values can be linked to the burst pressures or “seal breaking pressure.” A higher radial outward force will allow the physician to go to higher infusion pressures. In a presently preferred embodiment, the distal frame has the same radial outward force as the proximal frame. However, in an alternative embodiment, the distal frame has a lower radial outward force than the proximal frame as a safety feature, so that the drug would flow out the distal seal if the patient's blood vessel is over pressured. The frames are typically formed of a metal such as stainless steel or a NiTi alloy.

The membrane 15 is preferably a solid-walled, non-porous polymeric material to contain the agent within the agent containment pocket 32. A variety of suitable polymeric materials can be used to form the membrane 15 including polyurethanes, copolyamides such as polyether block amide (PEBAX) and styrenic block copolymers such as SYNPRENE, and a presently preferred membrane 15 is a polyurethane. Although discussed primarily in terms of delivery of an agent through an agent delivery lumen of the shaft, the membrane 15 can additionally or alternatively be used for agent delivery, by impregnating or coating the membrane (e.g., the outer surface thereof) with an agent which will elute from the membrane while the device is deployed in the patient's body lumen.

The catheter shaft, or other components, preferably do not extend along or otherwise obstruct the outer surface of the central section of the membrane 15. As a result, the outer surface of the central section of the membrane 15 between the deployed frames extends directly adjacent to the inner surface of the body lumen wall, such that the containment pocket 32 extends fully around an inner circumference of the body lumen (see FIGS. 5 and 10). Additionally, the outer surface of the membrane 15 between the deployed frames preferably is not radially forced against the inner surface of the wall of the body lumen 30, and is thus radially spaced from the inner surface of the body lumen wall so that the agent containment pocket 32 is formed whether agent is flowing or not.

A variety of suitable agents can be delivered using the catheter(s) and method(s) of the invention. The agents are typically intended for treatment and/or diagnosis of coronary, neurovascular, and/or other vascular disease, and may be useful as a primary treatment of the diseased vessel, or alternatively, as a secondary treatment in conjunction with other interventional therapies such as angioplasty or stent delivery. Suitable therapeutic agents include, but are not limited to, thrombolytic drugs, anti-inflammatory drugs, anti-proliferative drugs, drugs restoring and/or preserving endothelial function, and the like. A variety of bioactive agents can be used including but not limited to peptides, proteins, oligonucleotides, cells, and the like. A variety of diagnostic agents can be used according to the present invention. According to the present invention, agents described herein may be provided in a variety of suitable formulations and carriers including liposomes, polymerosomes, nanoparticles, microparticles, lipid/polymer micelles, and complexes of agents with lipid and/or polymers, and the like.

The dimensions of catheter 10, 50, 60 depend upon factors such as the catheter type and the size of the artery or other body lumen through which the catheter must pass. By way of example, the shaft 11 inner tubular member 16 typically has an outer diameter of about 0.022 to about 0.035 inch (0.56 to 0.87 mm), and outer member 17 typically has an outer diameter of about 0.060 to about 0.0785 inch (1.5 to 2.0 mm) and a wall thickness of about 0.004 to about 0.008 inch (0.10 to 0.20 mm). The central section of the membrane 15 between the deployed frames 13, 14 has a length of about 5.0 to about 30 mm, preferably about 10 to about 25 mm. The proximal and distal sections of the membrane (on the frames) are typically about 7.0 to about 11.0 mm in length, and the total length of the each frame from the proximal to the distal skirt sections is typically about 11.0 to about 13.5 mm. For example, in one embodiment, the membrane total length is about 25 mm, and the central section between the frames is about 10 mm long, and the frames together span a distance of about 32 mm. Typically, for coronary arteries, the frames radially expand to a maximum outer diameter of about 3.5 to about 4.5 mm. The overall length of the catheter 10, 50, 60 may range from about 100 to about 150 cm, and is typically about 143 cm.

The shaft tubular members can be formed by conventional techniques, for example by extruding and necking materials already found useful in intravascular catheters such a polyethylene, polyvinyl chloride, polyesters, polyamides, polyimides, polyurethanes, and composite materials. The various components may be joined using conventional bonding methods such as by fusion bonding or use of adhesives. A variety of suitable shaft configurations can be used including one or more of the tubular members formed of single or multiple layers or sections of tubing, as are conventionally known for catheter shaft design

While the present invention is described herein in terms of certain preferred embodiments, those skilled in the art will recognize that various modifications and improvements may be made to the invention without departing from the scope thereof. Moreover, although individual features of one embodiment of the invention may be discussed herein or shown in the drawings of the one embodiment and not in other embodiments, it should be apparent that individual features of one embodiment may be combined with one or more features of another embodiment or features from a plurality of embodiments.

We claim:

1. A catheter for delivering an agent to an inner surface of a patient's body lumen wall, comprising:
a) an elongated shaft having a distal shaft section and an agent delivery lumen which is in fluid communication with an agent delivery distal port in the distal shaft section;
b) a proximal frame and a distal frame on the distal shaft section which reversibly radially expand from a collapsed to a radially expanded configuration, each frame having a proximal end, a distal end, at least one skirt section mounting the frame on the distal shaft section, and a plurality of struts forming a body with a conical end section; and
c) a tubular membrane extending from the proximal to the distal frame, having a proximal end section secured to the proximal frame, a distal end section secured to the distal frame, a central section therebetween, an outer surface, and an inner surface defining a perfusion channel extending in the tubular membrane with the frames in the expanded configuration, and which has the shaft extending in the perfusion channel, and the agent delivery lumen extends across the membrane from the inner towards the outer surface thereof, so that the catheter is configured to deliver an agent to an agent containment pocket which extends along the inner surface of the patient's body lumen wall along an outer surface of the central section of the membrane.

2. The catheter of claim 1 wherein frames are radially self-expanding frames which reversibly radially self-expand to the expanded configuration upon removal of a radially restraining force.

3. The catheter of claim 1 wherein the agent delivery lumen is the only fluid delivery lumen in the shaft.

4. The catheter of claim 1 wherein each frame has a self-expansive force sufficient to fully open the frame to a maximum radially expanded outer diameter of the frame.

5. The catheter of claim 1 wherein the proximal frame has a radially expansive force different from the distal frame.

6. The catheter of claim 1 wherein the conical end section of each frame is a proximal conical end section.

7. The catheter of claim 1 wherein the conical end section of each frame extends along the central section of the membrane.

8. The catheter of claim 7 wherein the membrane central section is radially spaced from the frame conical end sections extending therealong in the expanded configuration, such that the perfusion channel has an inner diameter along the agent containment pocket that is larger than an outer diameter of the conical end sections of the frame.

9. The catheter of claim 7 wherein the frames share a common intermediate skirt section mounted on the shaft.

10. The catheter of claim 9 wherein the intermediate skirt is slidably mounted on the shaft.

11. The catheter of claim 1 wherein the membrane is bonded to an outer surface of each of the proximal and the distal frames.

12. The catheter of claim 1 wherein the membrane is contoured with a smaller outer diameter along the central section than along the proximal and/or distal end sections of the membrane in the expanded configuration.

13. A method of performing a medical procedure, comprising
a) introducing within a patient's body lumen a catheter comprising
i) an elongated shaft having a distal shaft section and an agent delivery lumen which is in fluid communication with an agent delivery distal port in the distal shaft section;
ii) a proximal self-expanding frame and a distal self-expanding frame on the distal shaft section which reversibly radially self-expand from a collapsed to a radially expanded configuration upon removal of a radially restraining force, each frame having a proximal end, a distal end, at least one skirt section mounting the frame on the distal shaft section, and a plurality of struts forming a body with a conical end section; and
iii) a tubular membrane extending from the proximal to the distal frame, having a proximal end section secured to the proximal frame, a distal end section secured to the distal frame, a central section therebetween, an outer surface, and an inner surface defining a perfusion channel extending in the tubular membrane with the frames in the expanded configuration, and which has the shaft extending in the perfusion channel, and the agent delivery lumen extends across the membrane from the inner towards the outer surface thereof;
b) radially self-expanding each frame by removing the radially restraining force therefrom to thereby force the proximal and distal end sections of the membrane against an inner surface of the patient's body lumen; and
c) delivering an agent from the agent delivery lumen to an agent containment pocket which extends along the inner surface of the patient's body lumen wall along an outer surface of the central section of the membrane.

14. The method of claim 13 wherein radially self-expanding the frames in b) opens the membrane such that the outer surface of the central section of the membrane is radially spaced from the inner surface of the patient's body lumen wall.

15. The method of claim 13 wherein the membrane is radially spaced from the conical end sections of the frames along the agent containment pocket, such that radially self-expanding the frames in b) opens the perfusion channel through the membrane having an inner diameter along the agent containment pocket that is larger than an outer diameter of the conical end sections of the frame.

16. The method of claim 13 wherein one of the proximal or distal frame has a lower radially expansive force than the other frame to thereby release agent above a seal-breaking pressure, and including preventing or reducing over pressurization of the patient's body lumen by releasing agent from the agent containment pocket.
